(19) United States
(12) Patent Application Publication
(10) Pub. No.: US 2015/0073454 A1
(43) Pub. Date: Mar. 12, 2015

(54) LIQUID EJECTION DEVICE AND MEDICAL APPARATUS

(71) Applicant: SEIKO EPSON CORPORATION, Tokyo (JP)

(72) Inventors: Kazuaki UCHIDA, Fujimi-machi (JP); Atsuya HIRABAYASHI, Chino-shi (JP); Hirokazu SEKINO, Chino-shi (JP)

(21) Appl. No.: 14/483,545

(22) Filed: Sep. 11, 2014

(30) Foreign Application Priority Data
Sep. 11, 2013 (JP) 2013-188265

(51) Int. Cl. A61B 17/3203; B05B 12/06

(52) U.S. Cl. CPC A61B 17/3203; B05B 12/06; A61B 2017/32032; A61B 2017/00973
USPC 606/167; 239/101

(57) ABSTRACT

A liquid ejection device includes: a liquid chamber with a variable inside volume; a volume varying unit which varies the volume inside the liquid chamber according to a drive signal; a volume changing unit which changes a representative volume of the liquid chamber; an ejection tube having an ejection port for ejecting a liquid from the liquid chamber; and a control unit which controls the volume varying unit and the volume changing unit and thereby adjusts pressure inside the liquid chamber. The control unit changes the representative volume according to a moving speed of the ejection port.

This application claims the benefit of Japanese Patent Application No. 2013-188265, filed on Sep. 11, 2013. The content of the aforementioned patent application is incorporated by reference herein in its entirety.

### BACKGROUND

1. Technical Field

The present invention relates to ejection of a liquid.

2. Related Art

In a liquid ejection device used as a medical apparatus, a technique of measuring the acceleration of an ejection port and selecting a liquid ejection mode based on the acceleration is know (see, for example, JP-A-2012-143374).

The related-art technique is an excellent technique that achieves an incision or excision ability as intended by the operator, by switching the ejection mode according to the moving speed of the ejection port. The inventors have improved this technique and have found out a method for suitably changing the incision ability or the like according to the moving speed of the ejection port. Also, miniaturization of the device, lower cost, saving of resources, easier manufacturing, improved user-friendliness and the like are demanded. The inventors have also tried solutions to these problems.

### SUMMARY

An advantage of some aspects of the invention is to solve at least one of the problems described above, and the invention can be implemented as the following forms.

(1) An aspect of the invention provides a liquid ejection device. The liquid ejection device includes: a liquid chamber with a variable inside volume; a volume varying unit which varies the volume inside the liquid chamber according to a drive signal; a volume changing unit which changes a representative volume of the liquid chamber; an ejection tube having an ejection port for ejecting a liquid from the liquid chamber; and a control unit which controls the volume varying unit and the volume changing unit and thereby adjusts pressure inside the liquid chamber. The control unit changes the representative volume according to a moving speed of the ejection port. According to this configuration, the representative volume, which is related to the excision depth, is changed according to the moving speed of the ejection port. Therefore, the excision ability can be adjusted according to the moving speed of the ejection port. The representative volume refers to a volume value at which it can be assumed that the influence of volume variation by the volume varying unit is eliminated.

(2) In the aspect described above, the control unit may set the representative volume to a first volume if the moving speed is a first speed, and may set the representative volume to a second volume that is smaller than the first volume if the moving speed is a second speed that is higher than the first speed. According to this aspect, the representative volume is changed in such a way that the excision ability is enhanced as the moving speed increases. Therefore, the excision depth can be stabilized.

(3) In the aspect described above, the control unit may set the representative volume to a third volume that is smaller than the second volume if the moving speed is a third speed that is higher than the second speed and if the moving speed is a fourth speed that is higher than the third speed. The control unit may change a frequency of the drive signal between the case of the third speed and the case of the fourth speed. According to this aspect, the drive frequency, which influences the excision depth, is changed according to the moving speed. Therefore, the excision ability can be adjusted according to the moving speed of the ejection port.

(4) In the aspect described above, the control unit may set a maximum voltage of the drive signal to a first voltage if the frequency of the drive signal is a first frequency, and may set the maximum voltage of the drive signal to a second voltage that is higher than the first voltage if the frequency of the drive signal is a second frequency that is higher than the first frequency. According to this aspect, the excision depth can be adjusted by changing the maximum voltage.

(5) In the aspect, the control unit may set the frequency of the drive signal to the second frequency and set the maximum voltage of the drive signal to the second voltage if the moving speed is the fourth speed. The control unit may set the frequency of the drive signal to the second frequency and set the maximum voltage of the drive signal to a third voltage that is higher than the second voltage if the moving speed is a fifth speed that is higher than the fourth speed. According to this aspect, the excision depth can be controller more easily. Since the frequency is not changed while the maximum voltage is changed between the case of the fourth speed and the case of the fifth speed, the influence of the maximum voltage and the influence of the frequency can be separated. Consequently, a proper maximum voltage and frequency can be determined more easily with respect to the moving speed. Therefore, the excision depth can be controlled more easily.

(6) Another aspect of the invention provides a liquid ejection device. The liquid ejection device includes: a liquid chamber with a variable inside volume; a volume varying unit which varies the volume inside the liquid chamber according to a drive signal; a volume changing unit which changes a representative volume of the liquid chamber; an ejection tube having an ejection port for ejecting a liquid from the liquid chamber; and a control unit which controls the volume varying unit and the volume changing unit and thereby adjusts pressure inside the liquid chamber. The control unit changes the representative volume and at least one of a frequency and a maximum voltage of the drive signal, according to a moving speed of the ejection port. According to this configuration, at least one of the frequency and the maximum voltage of the drive signal as well as the representative volume is changed according to the moving speed of the ejection port. Therefore, the excision ability can be adjusted according to the moving speed of the ejection port.

The invention can also be implemented in various other forms than the above. For example, the invention can be implemented in the form of a liquid ejection method, a medical apparatus, a surgical operation method, a program for realizing these methods, a storage medium storing the program, or the like.

### BRIEF DESCRIPTION OF THE DRAWINGS

The invention will be described with reference to the accompanying drawings, wherein like numbers reference like elements.

FIG. 1 shows the configuration of a liquid ejection device.

FIG. 2 shows the internal structure of a liquid ejection mechanism.

FIG. 3 is a graph showing a drive waveform.

FIG. 4 is a flowchart showing ejection processing (Embodiment 1).

FIGS. 5A to 5C are graphs showing the relation between each parameter and moving speed.

FIG. 6 is a graph showing how a drive waveform changes.

FIG. 7 is a graph showing the relation between peak voltage and drive frequency.

FIG. 8 is a flowchart showing ejection processing (Embodiment 2).

### DESCRIPTION OF EXEMPLARY EMBODIMENTS

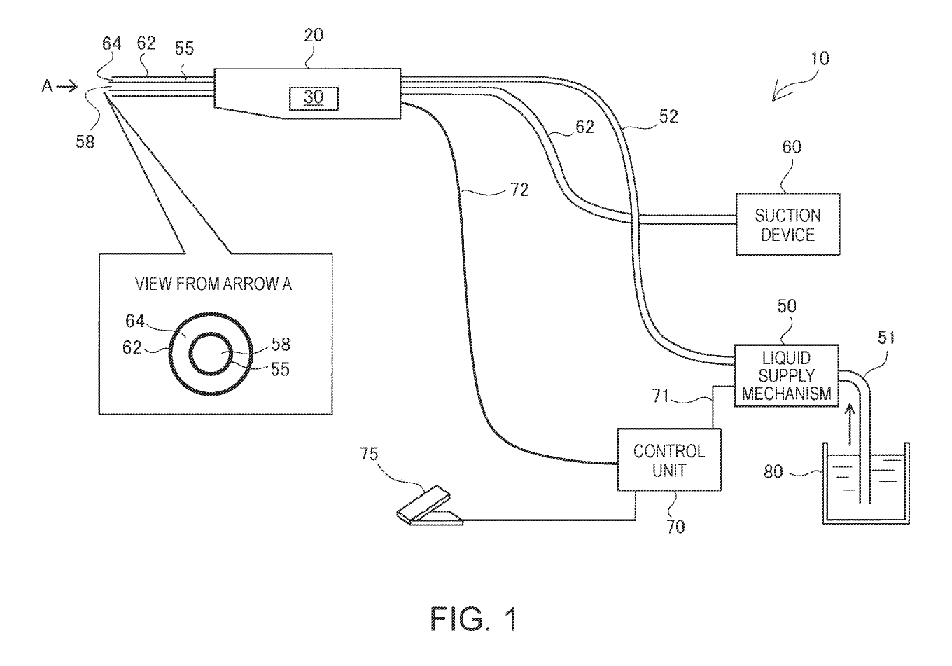

Embodiment 1 will be described. FIG. 1 shows the configuration of a liquid ejection device 10. The liquid ejection device 10 is a medical apparatus used in a medical institution and has the function of incising or excising an affected part by ejecting a liquid to the affected part.

The liquid ejection device 10 has a liquid ejection mechanism 20, a liquid supply mechanism 50, a suction device 60, a control unit 70, and a liquid container 80. The liquid supply mechanism 50 and the liquid container 80 are connected to each other via a connection tube 51. The liquid supply mechanism 50 and the liquid ejection mechanism 20 are connected to each other via a liquid supply channel 52. The connection tube 51 and the liquid supply channel 52 are made of a resin. The connection tube 51 and the liquid supply channel 52 may also be made of other materials than resin (for example, a metal).

The liquid container 80 stores a physiological saline solution. The liquid container 80 may store pure water or liquid medicine instead of the physiological saline solution. The liquid supply mechanism 50 supplies a liquid sucked from the liquid container 80 via the connection tube 51, to the liquid ejection mechanism 20 via the liquid supply channel 52, by driving a built-in pump.

The liquid ejection mechanism 20 is an instrument which the user of the liquid ejection device 10 holds in a hand to operate. The user applies the liquid intermittently ejected from an ejection port 58 to an affected part and thus incises or excises the affected part.

The control unit 70 transmits a drive signal to a pulsation generator 30 arranged inside the liquid ejection mechanism 20, via a signal cable 72. The control unit 70 controls the liquid supply mechanism 50 via a control cable 71 and thereby controls the flow rate of the liquid supplied to the pulsation generator 30. A foot switch 75 is connected to the control unit 70. As the user turns on the foot switch 75, the control unit 70 controls the liquid supply mechanism 50 to execute supply of the liquid to the pulsation generator 30 and also transmits a drive signal to the pulsation generator 30 to generate pulsation in the pressure of the liquid supplied to the pulsation generator 30. The mechanism for the generation of pulsation and the control of the ejection of the liquid from the liquid ejection mechanism 20 will be described in detail later.

The suction device 60 is for sucking the liquid and excised matter from around the ejection port 58. The suction device 60 and the liquid ejection mechanism 20 are connected to each other via a suction channel 62. The suction device 60 constantly sucks the inside of the suction channel 62 while a switch for operating the suction device 60 is on. The suction channel 62 penetrates the inside of the liquid ejection mechanism 20 and opens near the forward end of an ejection tube 55.

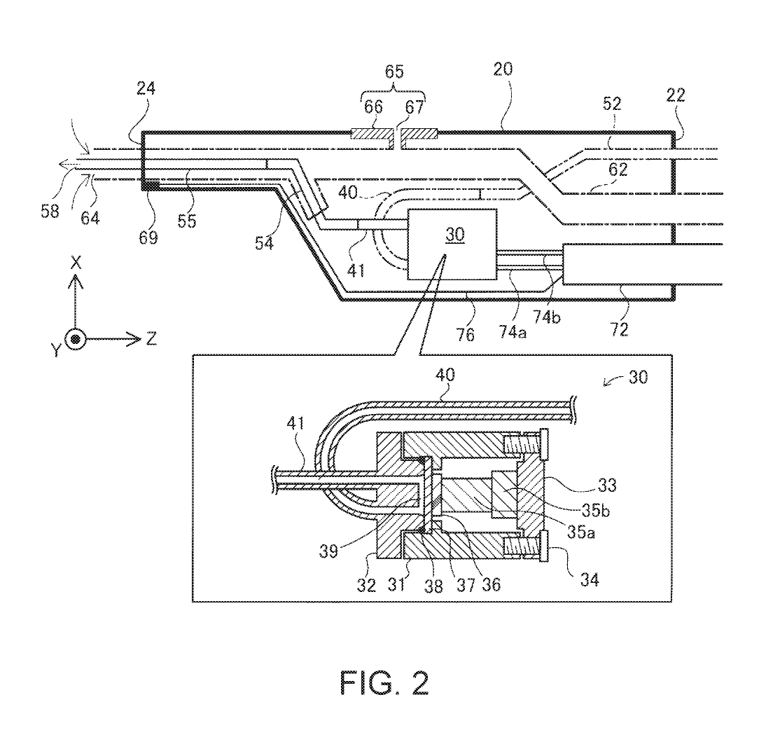

The suction channel 62 covers the ejection tube 55 extending from the forward end of the liquid ejection mechanism 20. Therefore, as shown in the view from arrow A in FIG. 1, the wall of the ejection tube 55 and the wall of the suction channel 62 form substantially concentric cylinders. A channel through which a sucked matter sucked from a suction port 64 that is the forward end of the suction channel 62 flows, is formed between the outer wall of the ejection tube 55 and the inner wall of the suction channel 62. The sucked matter is sucked by the suction device 60 via the suction channel 62. This suction is adjusted by a suction force adjustment mechanism 65, described later with reference to FIG. 2.

FIG. 2 shows the internal structure of the liquid ejection mechanism 20. The liquid ejection mechanism 20 has, inside thereof, the pulsation generator 30, an inlet channel 40, an outlet channel 41, a connection tube 54, and an acceleration sensor 69, and also has the suction force adjustment mechanism 65.

The pulsation generator 30 generates pulsation in the pressure of the liquid supplied to the liquid ejection mechanism 20 from the liquid supply mechanism 50 via the liquid supply channel 52. The liquid with the pulsating pressure is supplied to the ejection tube 55. The liquid supplied to the ejection tube 55 is intermittently ejected from the ejection port 58. The ejection tube 55 is made of a stainless steel. The ejection tube 55 may also be made of other materials having predetermined or higher rigidity, such as other metals like brass or reinforced plastics.

The pulsation generator 30 has a first case 31, a second case 32, a third case 33, a bolt 34, a first piezoelectric element 35a, a second piezoelectric element 35b, a reinforcement board 36, a diaphragm 37, a packing member 38, the inlet channel 40 and the outlet channel 41, as shown in the enlarged view at the bottom of FIG. 2. The first case 31 is a cylindrical member. As the second case 32 is connected to one end of the first case 31, and the third case 33 is fixed to the other end of the first case 31 with the bolt 34, the entire body is sealed. The first and second piezoelectric elements 35a, 35b are arranged in the space formed inside the first case 31.

The first and second piezoelectric elements 35a, 35b are multilayer piezoelectric elements. The first and second piezoelectric elements 35a, 35b are connected in series, with ends thereof in the stacking direction bonded to each other. The other end of the first piezoelectric element 35a is fixed to the diaphragm 37 via the reinforcement board 36. The other end of the second piezoelectric element 35b is fixed to the third case 33. The diaphragm 37 is made of a metallic thin film. The peripheral edge of the diaphragm 37 is fixed to the first case 31 and sandwiched between the first case 31 and the second case 32. A liquid chamber 39 is formed between the diaphragm 37 and the second case 32.

The signal cable 72 is inserted from a rear end part 22 of the liquid ejection mechanism 20. The signal cable 72 accommodates two electrode wires 74a, two electrode wires 74b, and one signal wire for acceleration sensor 76. The electrode wires 74a are connected to the first piezoelectric element 35a in the pulsation generator 30. The electrode wires 74b are connected to the second piezoelectric element 35b in the pulsation generator 30. The drive signal transmitted from the control unit 70 is transmitted to the first and second piezoelectric elements 35a, 35b via the electrode wires 74a, 74b in the signal cable 72. The first and second piezoelectric elements 35a, 35b expand and contract on the basis of the drive signal.

The liquid chamber 39 varies in volume due to the expansion and contraction of the first and second piezoelectric elements 35a, 35b. The first piezoelectric element 35a expands and contracts in order to eject the liquid intermittently. The second piezoelectric element 35b expands and contracts in order to vary a representative volume of the liquid chamber 39. The representative volume in this embodiment refers to the volume of the liquid chamber 39 in the state where no voltage is applied to the first piezoelectric element 35a.

The inlet channel 40, into which the liquid flows, is connected to the second case 32. The inlet channel 40 is curved in a U-shape and extends toward the rear end part 22 of the liquid ejection mechanism 20. The liquid supply channel 52 is connected to the inlet channel 40. The liquid supplied from the liquid supply mechanism 50 is supplied to the liquid chamber 39 via the liquid supply channel 52.

As the first piezoelectric element 35a expands and contracts at a predetermined frequency, the diaphragm 37 vibrates. As the diaphragm 37 vibrates, the volume of the liquid chamber 39 varies, causing pulsation in the pressure of the liquid in the liquid chamber. The pressurized liquid flows out from the outlet channel 41 connected to the liquid chamber 39.

The ejection tube 55 is connected to the outlet channel 41 via the metallic connection tube 54. The liquid flowing out to the outlet channel 41 is ejected from the ejection port 58 via the connection tube 54 and the ejection tube 55.

The suction force adjustment mechanism 65 is for adjusting the force with which the suction channel 62 sucks the liquid or the like from the suction port 64. The suction force adjustment mechanism 65 includes an operation part 66 and a hole 67. The hole 67 is a penetration hole that connects the suction channel 62 and the operation part 66 together. As the user opens and closes the hole 67 with a finger of the hand holding the liquid ejection mechanism 20, the amount of air flowing into the suction channel 62 via the hole 67 is adjusted according to the degree of the opening and closing. Thus, the suction force at the suction port 64 is adjusted. The adjustment of the suction force may also be realized by the control of the suction device 60.

The liquid ejection mechanism 20 has the acceleration sensor 69. The acceleration sensor 69 is a piezoresistive three-axis acceleration sensor. The three axes are the XYZ-axes shown in FIG. 2. The X-axis is parallel to the penetrating direction of the hole 67, where the upward direction is the positive direction. The Z-axis is parallel to the longitudinal direction of the ejection tube 55, where the direction in which the liquid is ejected is the negative direction. The Y-axis is defined by the right-hand system, based on the X-axis and Z-axis.

The acceleration sensor 69 is arranged near a forward end part 24 of the liquid ejection mechanism 20, as shown in FIG. 2. The result of measurement is inputted to the control unit 70 via the signal wire for acceleration sensor 76.

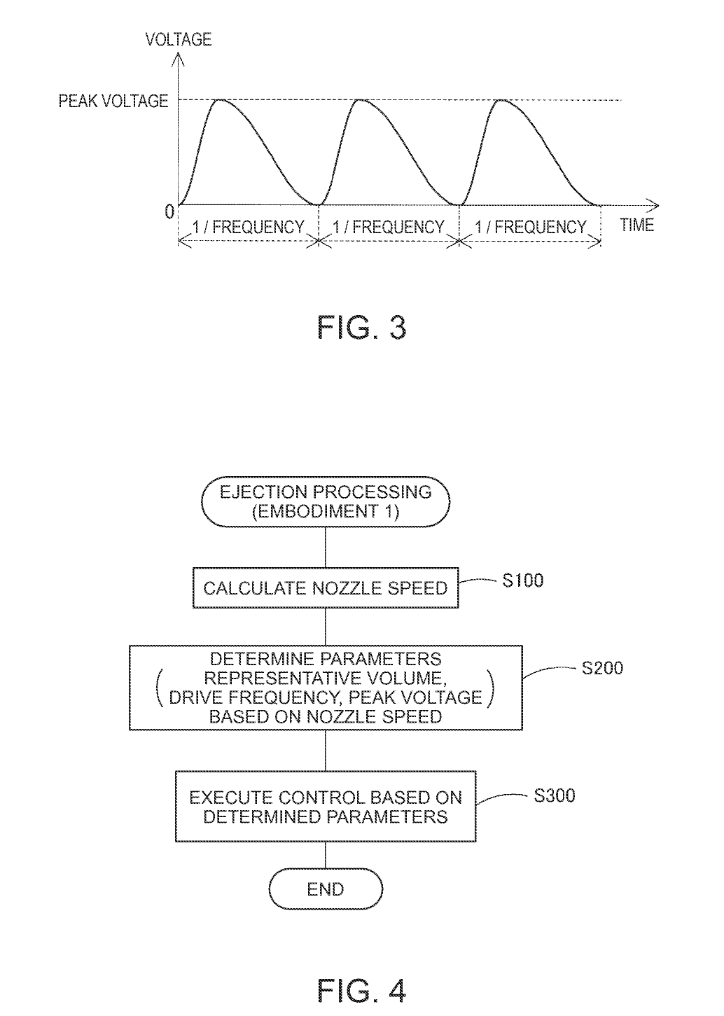

FIG. 3 is a graph showing the waveform of a drive signal inputted to the first piezoelectric element 35a (hereinafter referred to as “drive waveform”). The vertical axis represents voltage. The horizontal axis represents time. The drive waveform is drawn as a combination of sine curves. The peak voltage and frequency of the drive waveform change according to ejection processing (described later with reference to FIG. 4).

As the voltage value of the drive signal rises, the first piezoelectric element 35a is deformed in such a way that the volume of the liquid chamber 39 contracts. As the drive signal is inputted repeatedly, this contraction occurs repeatedly. Consequently, the liquid is ejected intermittently.

FIG. 4 is a flowchart showing the ejection processing. The ejection processing is executed repeatedly by the control unit 70 while the foot switch 75 is pressed down. First, a speed S of the ejection port 58 is calculated (Step S100). The speed S here refers to the absolute value of the speed in the XY plane. That is, the speed S is the absolute value of the speed ignoring the speed in the Z-axis direction. The speed S is calculated on the basis of the acceleration on the three axes measured by the acceleration sensor 69.

The speed S is calculated as a parameter that influences the excision depth at the affected part. This is because the excision ability acting on each local area of the affected part per unit time is influenced by the moving speed between the ejection port 58 and the affected part. The speed S may be treated as the moving speed between the affected part and the ejection port 58, considering that the affected part moves with the patient's breathing or the like. However, in this embodiment, the speed S is treated as the moving speed between the affected part and the ejection port 58 on the assumption that the affected part is static.

Next, each parameter is determined on the basis of the calculated speed S (Step S200). Control is executed on the basis of the determined parameters (Step S300). The parameters in this case are the representative volume, drive frequency and peak voltage.

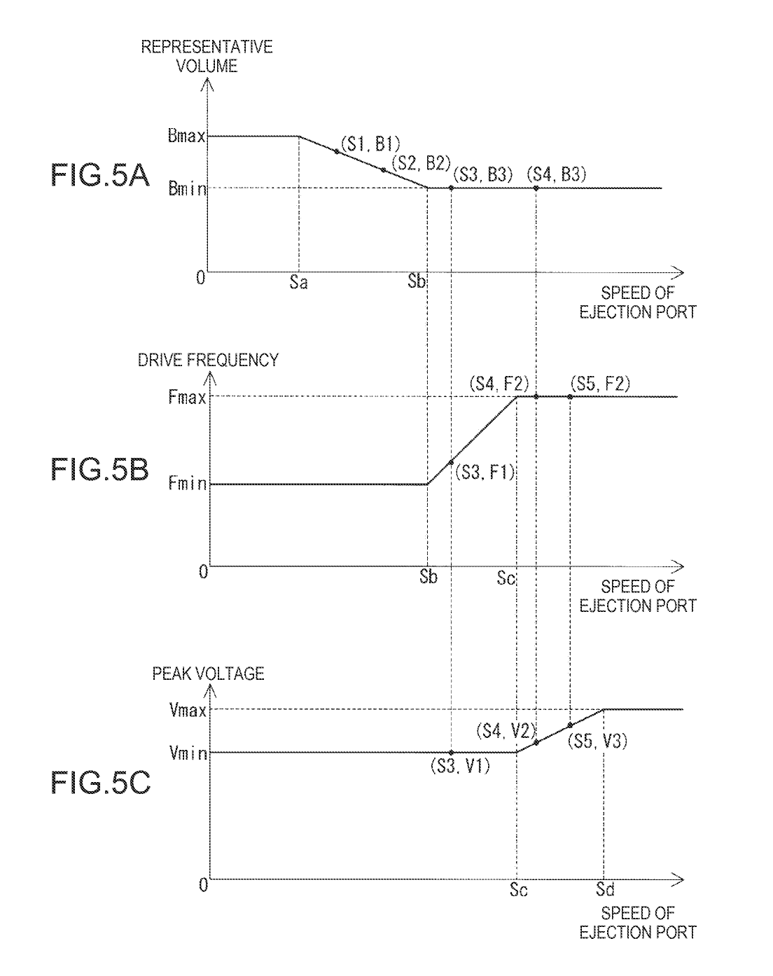

FIGS. 5A to 5C are graphs showing the relation between each parameter and the speed S. The vertical axis represents the representative volume in FIG. 5A, the drive frequency in FIG. 5B, and the peak voltage in FIG. 5C. The horizontal axis represents the speed S on the same scale across FIGS. 5A to 5C.

As shown in FIGS. 5A to 5C, the parameter that changes in value differ between the speed ranges of Sa≦the speed S≦Sb, Sb≦the speed S≦Sc, and Sc≦the speed S≦Sd. That is, Sa, Sb and Sc are speeds that are predetermined as thresholds at which the parameter to change is switched.

In the case of the speed S≦Sa, the representative volume is fixed at a maximum value Bmax, the drive frequency is fixed at a minimum value Fmin, and the peak voltage is fixed at a minimum value Vmin. When the parameters are set in this manner, the excision ability is at its minimum.

In the case of Sa≦the speed S≦Sb, the drive frequency is fixed at Fmin and the peak voltage is fixed at Vmin. Meanwhile, the representative volume linearly decreases with the increase in the speed S. In the case of the speed S=Sb, the representative volume is set to a minimum value Bmin.

The values Bmax and Bmin are set in such a way that an excessive force is not applied to the diaphragm 37. Bmin is also set under the condition that the expansion and contraction of the first piezoelectric element 35a does not cause the diaphragm 37 to contact the site facing the diaphragm, of the liquid chamber 39.

As the representative volume decreases, the variation ratio of volume variation by the first piezoelectric element 35a increases even if the amplitude of the volume variation is the same. The variation ratio refers to the value of the maximum volume divided by the minimum volume in the volume variation. As the ratio of the volume variation increases, pressure variation in the liquid chamber 39 increases. As the pressure variation in the liquid chamber 39 increases, the liquid is ejected with a greater force and therefore the excision ability rises. Consequently, even if the increase in the speed S causes a fall in the excision ability acting per unit area, the fall is offset and the excision depth is stabilized. The term “offset” here is not limited to the complete absence of change in the excision depth despite the change in the speed S, but includes at least partly canceling the influence of the change in the speed S.

In the case of Sb≦the speed S≦Sc, the representative volume is fixed at Bmin and the peak voltage is fixed at Vmin. Meanwhile, the drive frequency linearly increases with the increase in the speed S. In the case of the speed S=Sc, the drive frequency is set to a maximum value Fmax. Fmin is set in such a way that the excision ability is not too low and that intermittent ejection is realized. Fmax is set to a maximum frequency at which intermittent ejection is realized without lacking a sufficient flow rate of the liquid supplied from the liquid supply mechanism 50 to the liquid chamber 39. As the drive frequency is changed in this manner, the drive waveform changes.

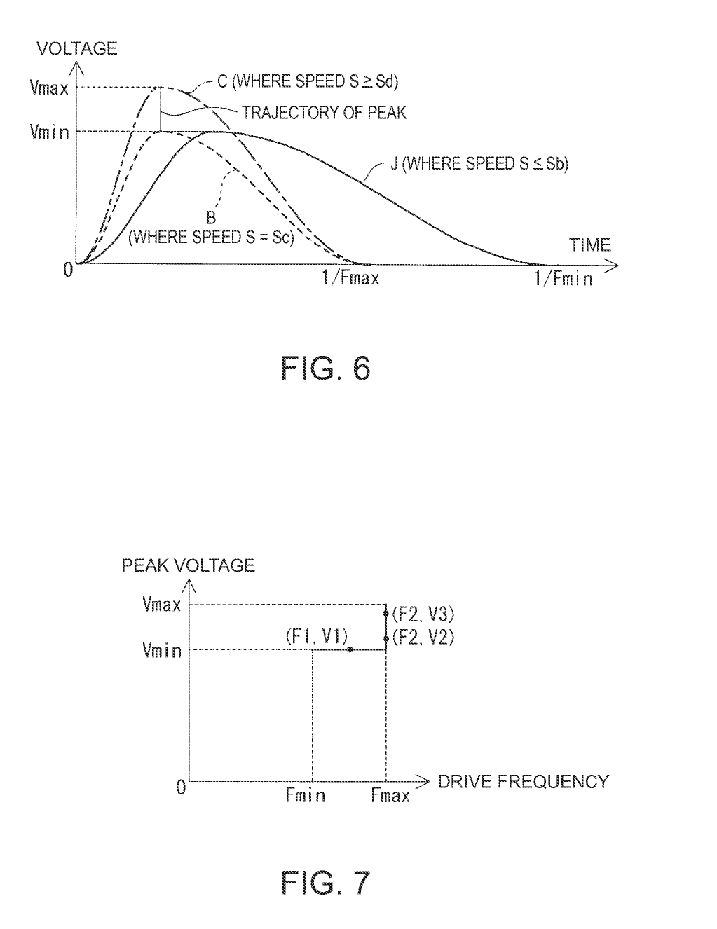

FIG. 6 is a graph showing how the drive waveform is changed. The vertical axis represents voltage. The horizontal axis represents time. FIG. 6 illustrates three drive waveforms. A curve J shows the drive waveform in the case where the drive frequency is set to Fmin and the peak voltage is set to Vmin. That is, the curve J shows the drive waveform in the above case of the speed S≦Sb. A curve B shows the drive waveform in the case where the drive frequency is set to Fmax and the peak voltage is set to Vmin. That is, the curve B shows the drive waveform in the above case of the speed S=Sc. A curve C shows the drive waveform in the case where the drive frequency is set to Fmax and the peak voltage is set to Vmax. That is, the curve C shows the drive waveform in the case of the speed S≦Sd, described below.

As the drive frequency increases, the number of times the liquid is ejected per unit time increases. Consequently, the excision ability rises and the excision depth is stabilized even if the speed S increases. Moreover, in the case of this embodiment, as the drive frequency increases, a rise time decreases, as shown in FIG. 6. The rise time refers to the time taken for the voltage value of the drive signal to reach a peak from zero. As the rise time decreases, the contraction of the liquid chamber 39 is executed in a shorter time. Consequently, the liquid is ejected with a greater force. Therefore, the excision ability rises and the excision depth is stabilized even if the speed S increases.

In the case of Sc≦the speed S≦Sd, the representative volume is fixed at Bmin and the drive frequency is fixed at Fmax. Meanwhile, the peak voltage linearly increases with the increase in the speed S. In the case of Sd≦the speed S, the peak voltage is fixed at Vmax, the representative volume is fixed at Bmin, and the drive frequency is fixed at Fmax. Vmin is set in such a way that the excision ability does not become too low. Vmax is set in such a way that the load on the first piezoelectric element 35a does not become too high.

As the peak voltage increases, the ratio of the volume variation increases. As the ratio of the volume variation increases, the excision ability rises and the excision depth is stabilized even if the seed S increases, as described above.

As described above, by changing the representative volume in such a way that the excision ability is enhanced as the speed S increases, the excision depth can be stabilized. Moreover, when the representative volume reaches the maximum value, the excision depth can be stabilized by changing the drive frequency and the peak voltage.

According to this embodiment, since the ranges of the speed S where the respective parameters (representative volume, drive frequency and peak voltage) are changed are separated, the value of each parameter can be determined easily in each speed range. Also, since the ranges of the speed S where the drive frequency is changed and where the peak voltage is changed are separated, the peak point of the drive waveform follows an L-shaped trajectory as shown in FIG. 6 when the drive waveform changes.

FIG. 7 is a graph showing the relation between peak voltage and drive frequency. As described already, the peak voltage does not change in the range of the speed S where the drive frequency changes, and the peak voltage changes in the range of the speed S where the drive frequency is set to Fmax.

S1 to S5 shown in FIGS. 5A to 5C and FIG. 7 are an example of the first to fifth speeds in the appended claims. B1 to B3 are an example of the first to third volumes. F1 and F2 are an example of the first and second frequencies. V1 to V3 are an example of the first to third voltages. The first piezoelectric element 35a and the diaphragm 37 in the embodiment are an example of the volume varying unit in the appended claims.

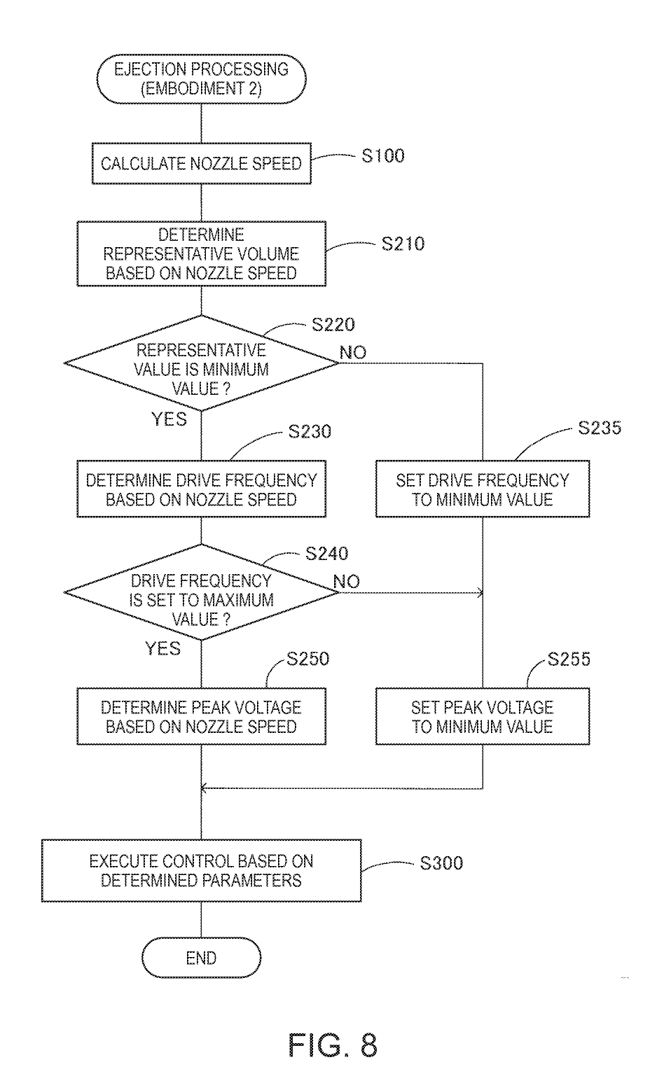

Embodiment 2 will be described. In Embodiment 2, the ejection processing shown in FIG. 8 is executed instead of the ejection processing shown in FIG. 4. The hardware configuration is the same as in Embodiment 1 and therefore will not be described further. Step S100 and Step S300 in the ejection processing of Embodiment 2 are the same as in Embodiment 1 and therefore will not be described further. In Embodiment 2, Steps S210 to S255 are executed instead of Step S200 of Embodiment 1.

After the speed S is calculated (Step S100), the representative volume is determined on the basis of the calculated speed S (Step S210). The technique for determining the representative volume is the same as in Embodiment 1. Next, whether the representative volume is set to the minimum value (Bmin) or not is determined (Step S220).

If the representative volume is set to a value that is not the minimum value (Step S220, NO), the drive frequency is set to the minimum value (Fmin) (Step S235) and the peak voltage is set to the minimum value (Vmin) (Step S255). The fact that the representative volume is set to a value that is not the minimum value means that there is some room for improvement in the excision ability by changing the representative volume. Therefore, since there is no need to change the drive frequency or the peak voltage to improve the excision ability, the drive frequency and the peak voltage are set to the minimum values.

Meanwhile, if the representative volume is set to the minimum value (Step S220, YES), the drive frequency is determined on the basis of the speed S (Step S230). The fact that the representative volume is set to the minimum value means that there is no room for improvement in the excision ability by changing the representative volume. Thus, Step S230 is executed in order to improve the excision ability by changing the value of the drive frequency.

Next, whether the drive frequency is set to the maximum value or not is determined (Step S240). If the drive frequency is set to a value that is not the maximum value (Step S240, NO), the peak voltage is set to the minimum value (Step S255). Meanwhile, if the drive frequency is set to the maximum value (Step S240, YES), the peak voltage is determined on the basis of the speed S (Step S250). The peak voltage is determined in this manner for a similar reason to the case where the drive frequency is set on the basis of the relation with the representative volume. Embodiment 2 can achieve the same control effects as Embodiment 1.

The invention is not limited to the embodiments, examples and modifications described herein and can be realized with various other configurations without departing from the spirit and scope of the invention. For example, the technical features of the embodiments, examples and modifications corresponding to the technical features of each configuration described in summary of the invention can be properly replaced or combined in order to solve a part or the whole of the foregoing problems or in order to achieve a part or the whole of the foregoing advantages. The technical features can be properly deleted if these features are not described as essential herein. For example, the following examples can be given.

The drive waveform may not be changed. That is, the adjustment of the excision ability may be realized by changing the representative volume only. If the configuration without changing the drive waveform is employed, the configuration of the control unit can be simplified.

The representative volume may be any value at which it can be assumed that the influence of the volume variation by the first piezoelectric element is eliminated. For example, this value may be the average value or maximum value of the volumes in the volume variation by the first piezoelectric element.

The technique for changing the representative volume may be other than using the second piezoelectric element. For example, another member may be inserted in the liquid chamber and the insertion depth thereof may be adjusted to change the representative volume. Alternatively, a piezoelectric element may be arranged at the site facing the diaphragm, in the liquid chamber, and the piezoelectric element may be made to expand and contract to change the representative volume.

Two piezoelectric elements arranged concentrically in a double structure may be used to change the representative volume. As a specific structure, for example, the inner piezoelectric element may be accommodated in a pot suspended from the top edge of the outer piezoelectric element. According to this structure, even if the outer piezoelectric element is expanded to push up the diaphragm, the distance between the inner piezoelectric element and the diaphragm remains unchanged. Therefore, the inner piezoelectric element may function as a volume varying unit and the outer piezoelectric element may function as a volume changing unit.

In the configuration using the double structure of the piezoelectric elements, the function of the outer piezoelectric element and the function of the inner piezoelectric element may be switched.

The representative volume, the drive frequency and the peak voltage may be determined, using a function.

The speed range where the representative volume is varied and the speed range where the drive frequency is varied may overlap with each other. The speed range where the drive frequency is varied and the speed range where the peak voltage is varied may overlap with each other. Alternatively, the speed range where the drive frequency is varied and the speed range where the peak voltage is varied may overlap with the speed range where the representative volume is varied.

The drive waveform may not be a combination of sine curves and may be increase or decrease stepwise, for example.

The relation between each of the representative volume, the drive frequency and the peak voltage, and the speed of the ejection port, may be prescribed curvilinearly or stepwise.

The drive frequency may be changed while the rise time is fixed. That is, the drive frequency may be changed by changing the time taken for the voltage of the drive signal to reach zero from the peak. Thus, the influence of the change in the rise time can be eliminated when determining the drive frequency with respect to the moving speed. Therefore, the drive frequency can be determined more easily.

The speed of the ejection port may be calculated, for example, by the acceleration sensor installed at the forward end of the ejection port. In this case, the result of the calculation is expected to be more accurate.

Alternatively, the speed of the ejection port may be calculated using image processing. For example, a marker may be placed at the forward end of the ejection port and the movement of the marker may be captured by a camera, thus calculating the speed of the ejection port.

If a robot operates the liquid ejection device, the robot can grasp the speed of the ejection port. Therefore, there is no need to calculate the speed and the grasped value may be used.

The moving speed of the ejection port may be calculated in consideration of the moving speed of the affected part. The measurement of the moving speed of the affected part may be realized by predicting or measuring the movement due to breathing or pulsation.

The acceleration sensor may be an electrostatic capacitance type or a heat detection type.

Also, a sensor capable of indirectly or directly detecting speeds may be used, other than the acceleration sensor.

The liquid ejection device may also be used for other purposes than medical apparatus.

For example, the liquid ejection device may be used for a cleaning apparatus which eliminates stains with an ejected liquid.

The liquid ejection device may also be used for a drawing apparatus which draws lines or the like with an ejected liquid.

A liquid ejection system using a laser beam may be employed. As an ejection system using a laser beam, for example, a laser beam may be intermittently cast on a liquid to gasify the liquid, and pressure variation generated by the gasification may be used.

What is claimed is:

1. A liquid ejection device comprising:
a liquid chamber with a variable inside volume;
a volume varying unit which varies the volume inside the liquid chamber according to a drive signal;
a volume changing unit which changes a representative volume of the liquid chamber;
an ejection tube having an ejection port for ejecting a liquid from the liquid chamber; and
a control unit which controls the volume varying unit and the volume changing unit and thereby adjusts pressure inside the liquid chamber;
wherein the control unit changes the representative volume according to a moving speed of the ejection port.

2. The liquid ejection device according to claim 1, wherein the control unit sets the representative volume to a first volume if the moving speed is a first speed, and sets the representative volume to a second volume that is smaller than the first volume if the moving speed is a second speed that is higher than the first speed.

3. The liquid ejection device according to claim 2, wherein the control unit sets the representative volume to a third volume that is smaller than the second volume if the moving speed is a third speed that is higher than the second speed and if the moving speed is a fourth speed that is higher than the third speed, and the control unit changes a frequency of the drive signal between the case of the third speed and the case of the fourth speed.

4. The liquid ejection device according to claim 3, wherein the control unit sets a maximum voltage of the drive signal to a first voltage if the frequency of the drive signal is a first frequency, and sets the maximum voltage of the drive signal to a second voltage that is higher than the first voltage if the frequency of the drive signal is a second frequency that is higher than the first frequency.

5. The liquid ejection device according to claim 4, wherein the control unit sets the frequency of the drive signal to the second frequency and sets the maximum voltage of the drive signal to the second voltage if the moving speed is the fourth speed, and the control unit sets the frequency of the drive signal to the second frequency and sets the maximum voltage of the drive signal to a third voltage that is higher than the second voltage if the moving speed is a fifth speed that is higher than the fourth speed.

6. A liquid ejection device comprising:
a liquid chamber with a variable inside volume;
a volume varying unit which varies the volume inside the liquid chamber according to a drive signal;
a volume changing unit which changes a representative volume of the liquid chamber;
an ejection tube having an ejection port for ejecting a liquid from the liquid chamber; and
a control unit which controls the volume varying unit and the volume changing unit and thereby adjusts pressure inside the liquid chamber;
wherein the control unit changes the representative volume and at least one of a frequency and a maximum voltage of the drive signal, according to a moving speed of the ejection port.

7. A medical apparatus using the liquid ejection device according to claim 1.

8. A medical apparatus using the liquid ejection device according to claim 2.

9. A medical apparatus using the liquid ejection device according to claim 3.

10. A medical apparatus using the liquid ejection device according to claim 4.

11. A medical apparatus using the liquid ejection device according to claim 5.

12. A medical apparatus using the liquid ejection device according to claim 6.
